(19) United States
(12) Patent Application Publication
(10) Pub. No.: US 2006/0051734 A1
(43) Pub. Date: Mar. 9, 2006

(54) Apparatus for mapping biological tissue quality

(76) Inventors: Stuart Alan McNeill, Edinburgh (GB); Robert Lewis Reuben, Balerno (GB)

Correspondence Address: Thomas N Young;Young & Basile, Suite 624, 3001 West Big Beaver Road, Troy, MI, 48084, US

(21) Appl. No.: 10/537,412

(22) PCT Filed: Dec. 3, 2003
(86) PCT No.: PCT/GB03/05217

§ 371 Date: Jun. 2, 2005

(30) Foreign Application Priority Data
Dec. 4, 2002 (GB) 0228276.2

(51) Int. Cl. C12M 1/34; C12Q 1/00

(52) U.S. Cl. 435/4; 435/287.1

(57) ABSTRACT

The present invention provides an apparatus suitable for use in investigating multi-phase biological tissue histology, which apparatus comprises a trans-ductally deployable probe mounting a periodically displaceable body of at least one tactile sensing device, said periodically displaceable body having an excitation frequency bandwidth in the range of from 1 Hz to 500 KHz, a maximum stroke length of less than 1 mm and a displacement force in the range from 0.01 N to 1 N, said displaceable body being provided with a displacement device having a displacement controller fro controlling at least said excitation frequency, said displaceable body being coupled to a displacement monitoring device and a displacement force monitoring device, for monitoring the viscoelastic response of said biological tissue to periodic compression by said displacement force applied to said tissue by periodic displacement of said periodically displaceable body. The present invention also includes a method for producing a histological profile of a biological tissue adjacent a body duct, and a diagnostic method, using the apparatus of the invention.

The present invention relates to the field of histological profiling of biological tissue and more specifically to an apparatus for histologically profiling tissue adjacent to body ducts.

Physicians use a number of steps when investigating and diagnosing a patient's condition including consideration of the patient's symptoms, results of clinical tests and the patient's response to various potential therapeutic regimes. For many conditions an accurate initial diagnosis can mean the patient is directed towards the most appropriate course of therapy particularly where a number of potential therapeutic options exist. This is beneficial for the patient who receives more rapid relief from the condition and reduces the likelihood of him undergoing a cascade of treatments in an attempt to match a therapy to the condition. It is also highly beneficial to health services for economic reasons, saving resources in terms of reducing the incidence of inappropriate therapies being prescribed and wasted man hours, as patients should pass through the system more quickly due to more rapid diagnosis and treatment, thereby reducing the number of return visits to the clinic.

A number of tissue abnormalities may cause patients to present with a classic characteristic pattern of symptoms. For example proliferation of different histological components of the prostate may cause patients to present with a classic characteristic pattern of symptoms indicating bladder outflow obstruction in benign prostate hyperplasia (BPH) or cancer of the prostate. The different histological components are thought to contribute to bladder outflow obstruction in different ways. By gaining an insight into the tissue make-up of an organ and any abnormalities of those tissue components, the root cause of certain conditions may be more accurately diagnosed and hence more effectively treated. Ideally a tool or apparatus for use in a procedure to ascertain this information would be minimally invasive, have a low risk for the patient and be simple and rapid to use.

One of the simplest techniques used to examine and investigate the consistency and substance of a tissue in proximity to body ducts relies on manual palpation of the tissue using a finger inserted into the body duct, for example, intra-rectally, intra-vaginally etc. Such a technique has obvious disadvantages in being highly subjective, having limitations in the depth of tissue which may be examined, and being restricted by how far up the duct a finger can reach.

A number of tools using ultrasound imaging, for example as described in U.S. Pat. No. 5,265,612 and U.S. Pat. No. 5,107,837, are known. However, these are normally directed at locating abnormalities in tissue revealed by differences in elastic properties compared to those of the matrix. Others (eg. U.S. Pat. No. 5,785,663) have used pressure distribution under a probe to reveal surface and sub-surface areas of differing elastic properties to that of the matrix tissue.

It is an object of the present invention to reduce or overcome one or more of the disadvantages of existing tools for use in investigating the histological make-up of biological tissue adjacent to body ducts.

In a first aspect the present invention provides an apparatus suitable for use in investigating multi-phase biological tissue histology, which apparatus comprises a trans-ductally deployable probe mounting a periodically displaceable body of at least one tactile sensing device, said periodically displaceable body having an excitation frequency bandwidth in the range of from 1 Hz to 500 KHz, a maximum stroke length of less than 1 mm and a displacement force in the range from 0.01 N to 1 N, said displaceable body being provided with a displacement device having a displacement controller for controlling said excitation frequency and stroke length, said displaceable body being coupled to a displacement monitoring device and a force monitoring device, for monitoring the viscoelastic response of said biological tissue to periodic compression by said displacement force applied to said tissue by periodic displacement of said periodically displaceable body.

Thus the present invention provides an apparatus that can monitor the compressibility of a tissue i.e. its springiness, but also the damping effect of a tissue, which together constitute the viscoelastic properties of the tissue. In the case of, for example, the prostate, muscular components have more spring-like characteristics while glandular components have a greater tendency to act as dampers. Certain materials, including most biological tissues, do not obey Hooke's Law but show viscoelastic properties which can be described by relating the time-varying stress σ(t) to strain history ε(t−Δt), as well as to the time-varying strain ε(t) according to the following equation:

$$σ ( t ) = {D}_{1} ɛ ( t ) - {\int }_{0}^{∞} [ \frac{{D}_{2}}{τ} {e}^{- Δ t / τ} ] ɛ ( t - Δ t ) \cdot ⅆ Δ t$$

where D₁ and D₂ are elastic properties of the material and τ is a characteristic time constant. When such a material is subjected to a sinusoidal strain ε=\hat{ε}sin ωt, the stress is given by:

$$σ ( t ) = ( {D}_{1} - \frac{{D}_{2}}{{ω}^{2} {τ}^{2} + 1} ) \hat{ɛ} cos ω t - {D}_{2} \frac{ωτ}{{ω}^{2} {τ}^{2} + 1} \hat{ɛ} sin ω t$$

When such materials are subjected to a sinusoidal strain, as in the periodic displacement of the displaceable body of the present invention compressing biological tissue, the strain and stress are related by:
σ=(E₁+iE₂)ε
where the quantity (E₁+iE₂), is a complex number corresponding to the dynamic modulus, which provides a full description of the material's elastic properties. Often the modulus of this complex number is referred to loosely as the dynamic modulus, and is measured in an experiment as the ratio of the amplitude of the stress to the amplitude of the strain, hereinafter referred to as |E*| or Amplitude Ratio.

It is often the case that over-proliferation of one or more tissue components, or an upset in the balance of particular tissue components, can lead to pathology and the apparatus of the present invention may be of some assistance in identifying and differentiating between such different scenarios.

It will be understood that trans-ductally deployable probes according to the present invention can be made in various different sizes and forms suitable for insertion into and along various different body ducts or conduits including, but not limited to, the genito-urinary tract in males and females, the gastro-intestinal tract, the respiratory tract, and within the arterial and venous vasculature system. In addition such a probe could also be incorporated into apparatus used for laparoscopic surgery to replace the tactile feedback that is otherwise missing.

In general the probe would be dimensioned so as to have a diameter such that it can be more or less readily accommodated within the body duct, whilst being a more or less close fit therein. Naturally there can be a significant variation in size of a given body duct from one patient to another. At the same time, most body ducts are more or less readily expandable to a greater or lesser degree between a substantially relaxed condition, which may be more or less fully collapsed, a normally dilated condition in which, for example, a normal fluid flow level passes through the duct, and a substantially dilated condition, in which a significantly greater degree of force than normal is applied to push the duct walls apart without, however, endangering their integrity. Thus for example, in the case of the adult male urethra, the average normal diameter would be of the order of 5 mm, but could range from 3.5 to 7 mm. The urethra of average dimensions could however have a diametral range from fully relaxed to substantially dilated of from 4 to 10 mm.

Typically an apparatus suitable for trans-urethral insertion would have a probe diameter of not more than 5 mm and a probe length generally in the range from 1 to 3 cm, preferably of around 15 mm. Such a probe would preferably be mounted at one end of an elongate, trans-ductally deployable deployment device typically of length 25 to 30 cm.

It will be appreciated that as the dimensions of the ducts of the gastro-intestinal system are much larger and more distensible they can accommodate endosocopes or probes with a significantly larger diameter, typically up to 12 to 20 mm. An apparatus according to the present invention may also be suitable for insertion into ducts of the respiratory or vasculature system. It will be appreciated that such systems comprise ducts whose diameters vary over a wide range. For example, the ducts of the respiratory tract progressively diminish in diameter from approximately 15 to 20 mm down to a few micrometres; similarly the larger vessels of the vasculature system are approximately 20 to 30 mm in diameter whilst the smaller capillaries have diameters in the micrometre range. The diameter of the probe can, therefore, be designed to have dimensions appropriate to those of the smallest ducts in which the probe is required to be used. For example, an apparatus with a probe diameter of 1.5 to 3 mm would be suitable for reaching the cardiac artery. It will be understood that the probe of such an apparatus would be mounted on an elongate deployment device of sufficient length and appropriate diameter to enable the probe to reach the ducts in question as the probe is inserted into the body and passed through the duct system thereof.

The periodically displaceable body is of substantially rigid and inert material selected from those which have sufficient durability to withstand repeated probing, vibrational displacement and the reciprocal force and pressure experienced from contacting and compressing surrounding tissue. Preferably the material should also be bio-compatible to reduce the likelihood of an adverse body reaction to the apparatus, such as an allergic or sensitivity response and the material should preferably be non-toxic. It is preferable for the material to be bio-resistant and able to withstand contact with biological fluids, chemicals, enzymes etc. encountered within the body when in use such as urea, amylase, hydrochloric acid. Suitable materials include suitable metals such as stainless steel, and natural or synthetic polymers including polyalkenes such as polyethylene, polypropylene, natural or synthetic rubber, including silicone rubber. Of course other materials could also be used as well if they are coated or sheathed in a suitable bio-compatible and bio-resistant material as discussed above.

Advantageously there is used a material which is of relatively low friction when in contact with body duct walling. Preferably or additionally the probe may be used with a physiologically acceptable lubricant, such as a water-based lubricating gel.

Other parts of the apparatus and probe which are inserted in and have contact with body components are generally also of bio-compatible and bio-resistant material.

It will be appreciated that the periodic displacement of the displaceable body can be actuated by a variety of means. For example, suitable pressurized fluid (hydraulic or pneumatic) circuits; mechanical systems, such as proximally mounted (at the exterior—outside the patient's body—end of the elongate probe deployment device) motors or distally-mounted motors provided with a suitable flexible drive-shaft and using devices such as cams, cranks and the like for converting rotary motion to reciprocating motion; and piezoelectric actuators.

Where pressurized fluid circuits are utilized it is convenient to have the pump or other pressurized fluid source external of the patient body being investigated when the apparatus is in use. The time varying displacement force and displacement can also be sensed and controlled externally of the patient body, thereby minimizing the apparatus bulk requiring to be inserted into the body.

It is convenient to provide the displacement by means of micro-pistons and shoes formed of a rigid material, such as silicon or nickel which can be micro-fabricated. The diameter of the micro-pistons would typically be around one fifth of the diameter of the probe. Hydraulic or pneumatic pressure or a suitable mechanical system of the type described above, for example, could be used to actuate the micro-pistons and shoes. Where a pressurized fluid circuit is used the force associated with such an actuator could be measured using the drive fluid pressure. Where a mechanical system is used a force transducer could be incorporated into the system to measure the displacement force. Such a force transducer could be micro-fabricated from a diaphragm of etched silicon incorporating piezo-resistive strain gauges and the piston suspended on this to measure the force. A number of systems also exist to measure displacement which will be apparent to those skilled in the art and include the use of interferometric measurement of distance using an optical fibre addressing some part of the displaceable body.

An apparatus incorporating piezoelectric-actuated displacement of the displaceable body is particularly advantageous because the piezoelectric device can be readily incorporated within the probe increasing compactness of the apparatus and ease of use. Generally any piezoelectric material with piezoelectric properties such as quartz, Rochelle salt, or, preferably, materials based on lead zirconate titanate (PZT) may be used. The piezoelectric material can be provided with a force transducer by mounting it onto a micro-fabricated diaphragm of etched silicon incorporating piezo-resistive strain gauges. Alternatively, the displacement may be provided by a mechanical system, such as a small-diameter electrostatic or electromagnetic rotary motor coupled to a mechanical system of the type identified above to provide reciprocating motion at the pistons or shoes. The current and voltage characteristics of the motor could then be used to deduce the actuation displacement force.

It will be appreciated that the range of displacement frequencies, displacement stroke lengths and displacement force values used to investigate the viscoelastic properties of different tissues may vary. For example those tissues with a denser structure and make-up which offer increased resistance to displacement by the displaceable body such as those with a higher collagen content should generally have a greater displacement force applied to them than less dense and more readily displaceable tissue, such as glandular material.

The range of suitable displacement frequency, displacement stroke length and displacement force for each type of tissue may be readily determined experimentally using a set of samples which contain a range of proportions of the histological components or other quality factor it is of interest to identify. For example, the probe might be useful to look for near-surface cancer, and the nature of the cancer (whether diffuse or focused, how far developed etc), or to assess the degree of calcification of arterial plaque.

The tactile sensing device is generally in close proximity to the tissue being compressed when the apparatus is in use, preferably in direct contact with the tissue. Conveniently the sensor of the tactile sensing device is incorporated within the displaceable body of the probe. Preferably the sensing device can detect the dynamic stress and dynamic strain experienced by the tissue being compressed. The measurements of the time-varying force and time-varying displacement are used to determine the time-varying stress and time-varying strain. According to the equations described hereinabove; namely

$$σ ( t ) = ( {D}_{1} - \frac{{D}_{2}}{{ω}^{2} {τ}^{2} + 1} ) \hat{ɛ} cos ω t - {D}_{2} \frac{ωτ}{{ω}^{2} {τ}^{2} + 1} \hat{ɛ} sin ω t ,$$

\hat{ε}=\hat{ε}sin ωt, and σ=(E₁+iE₂)ε, for each frequency of actuation, values of the components (E₁, E₂) of the dynamic modulus can be obtained from the ratio of amplitudes of the time-varying stresses and time-varying strains and the phase difference between the stress and strain. The variation of these components with frequency can then be used as the means of characterising the tissue being compressed. In practice these values of dynamic modulus would differentiate prostate tissue affected by BPH or cancer from a typically normal prostate.

Generally the displaceable body of the apparatus has a fixed contact surface area over which the displacement force is applied. However, in an alternative form of the present invention the dimensions of the displaceable body may be altered by simply manually interchanging alternative displaceable bodies of the probe. Alternatively the probe of the apparatus may be provided with an expandable and contractable displaceable body whose dimensions can be controlled by techniques well known in the art.

Many suitable force detectors may be incorporated within the displaceable body including etched silicon diaphragms with integral strain gauges. Alternatively force detectors may be incorporated within the displacement controller or at the force source. For example where hydraulic actuation is utilised the force experienced by the tissue being profiled can be simply determined from knowledge of the input force or the pressure in the hydraulic fluid.

It is convenient to use devices which are under displacement control, that is those of the type where the displacement is delivered by an actuator whose position is controlled and hence, the control signal for the actuator can be used to measure the dynamic displacement. Preferably said actuator is of rigid form. Convenient actuators include electromagnetic shakers or piston pumps (only suitable for use outside the body for acting on a hydraulic piston transmitting hydraulic fluid pressure to the interior of the duct via a suitable hydraulic fluid circuit), mini-rotary motors remotely coupled to a cam-lift mechanism, and piezoelectric crystals mounted in the probe for operating more or less directly on the tissue.

The apparatus is typically provided with a processing unit external to the body being investigated, which processes the input and output data to generate at least one of dynamic modulus, and Amplitude Ratio. Typically, a number of displacement cycles will be applied to a single site at each frequency, although a mixed frequency displacement could be applied to minimise the intervention. Measured values of stress and strain, as a function of time, will then be signal-averaged and the phase difference and amplitude ratio determined at each frequency, thus yielding a value of dynamic modulus for each frequency. The dynamic modulus will be referred to interchangeably with the complex modulus, herewithin.

By repeating the periodical displacement at a variety of different vibrational frequencies, a dynamic or complex modulus characteristic of the tissue can be generated. The displaceable body and probe may be easily moved to different positions at the surface of the tissue and the periodic displacement of the tissue repeated to analyse a plurality of regions of the tissue, providing a surface contour profile of the mechanical response. Such a profile may be generated by moving the displaceable body axially or circumferentially within the duct, or by providing a multiplicity of such displaceable bodies distributed axially and/or circumferentially.

The profile or map may be displayed via any suitable display device coupled to the apparatus such as a VDU screen or a printing or plotting device.

Preferably the apparatus is provided with a reference database of profiles or maps of tissues with known histologies against which comparison of subsequent tissue may be compared and assessed. Where such a facility is incorporated a computer generated description of the tissue histology may be provided, and possibly even some diagnostic indications.

It should be noted that whilst a full description of a material's viscoelastic properties is contained in the dynamic modulus, practically useful information is also obtainable from the Amplitude Ratio (|E*|), which is sometimes loosely (not correctly) also referred to as “dynamic modulus”. This Ratio is obtainable simply as the ratio of the amplitude of the stress to the amplitude of the strain at a given frequency, and is different at different frequencies—as a reflection of the “damping” component of the dynamic modulus.

It has been found that significant differences between benign and malignant tissue can be discerned by consideration of Amplitude Ratio (only) at a given frequency—typically in the range from 1 to 30 Hz. For the avoidance of doubt therefore, the present invention also encompasses methods and apparatus for monitoring Amplitude Ratio (only).

In a second aspect the present invention provides a method for producing a histological profile of a biological tissue adjacent a duct comprising the steps of:
a) providing an apparatus according to the present invention;
b) transductally inserting the probe of said apparatus to bring the periodically displaceable body of said probe into contact with the ductal surface of said biological tissue at a plurality of positions across said ductal surface;
c) subjecting said displaceable body to a periodic displacement at an excitation frequency bandwidth of from 1 Hz to 500 kHz, a maximum stroke of less than 1 mm and a displacement force in the range of from 0.01 N to 1 N so as to periodically compress said biological tissue at said contact positions across said ductal surface;
d) monitoring the viscoelastic response of said tissue at each of said surface contact tissue positions to compression by said body; and
e) generating a profile of the viscoelastic response of the tissue across said ductal surface.

Using the method of the present invention it is possible to obtain information on the viscoelasticity of a given tissue. This property of the tissue is tissue type dependent, some tissues being more compressible and springy while others have an increased damping effect when a force is removed and take longer to return to their uncompressed dimensions. The method according to the present invention can therefore be used to indicate the histological make up of the tissue.

In BPH for example such a method could assist a physician in diagnosing the histological abnormality causing bladder outflow obstruction and appropriate treatment be prescribed accordingly. For example, a prostate with an increased glandular content according to the viscoelastic profile could be treated with medication to reduce the bulk of the glandular material, such as 5-alpha-reductase inhibitors. A prostate where the muscle content is abnormal and fails to relax could be treated with muscle relaxants such as alpha-adrenergic blockers. Furthermore, by making use of differences in tissue characteristics arising from benign and malignant processes within the prostate, more effective use of resources can be achieved by correct identification of appropriate and cost effective treatments at an early stage.

In a further aspect the present invention provides a method of diagnosing a condition manifested by a histological abnormality in biological tissue adjacent a body duct comprising the steps of:
a) providing an apparatus according to the present invention;
b) trans-ductally inserting the probe of said apparatus to bring the periodically displaceable body of said probe into contact with the ductal surface of said biological tissue at successive ones of a plurality of positions across said ductal surface;
c) subjecting said displaceable body to a periodic displacement at an excitation frequency bandwidth of from 1 Hz to 500 kHz, a maximum stroke length of less than 1 mm and a displacement force in the range of from 0.01 N to 1 N so as to periodically compress said biological tissue at said contact positions across said ductal surface;
d) monitoring the viscoelastic response of said tissue at each of said tissue surface contact positions to compression by said body;
e) generating a profile of the viscoelastic response of the tissue across said ductal surface;
f) comparing said generated viscoelastic response profile with viscoelastic response profiles of such tissue having known histological characteristics.

Further preferred features and advantages of the invention will appear from the following examples and detailed description illustrated with reference to the accompanying drawings in which:

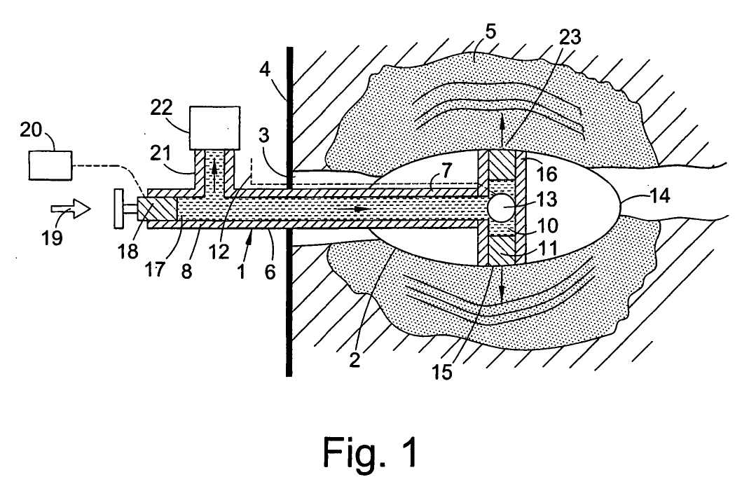

FIG. 1 is a schematic sectional view through one embodiment of a tissue quality measurement apparatus of the invention in use;

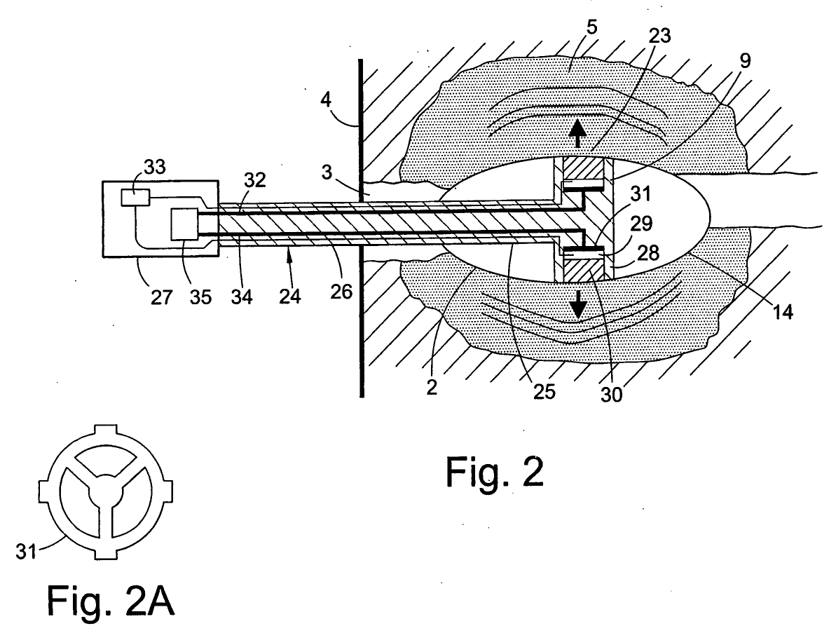

FIGS. 2. and 3 are corresponding views of two further embodiments;

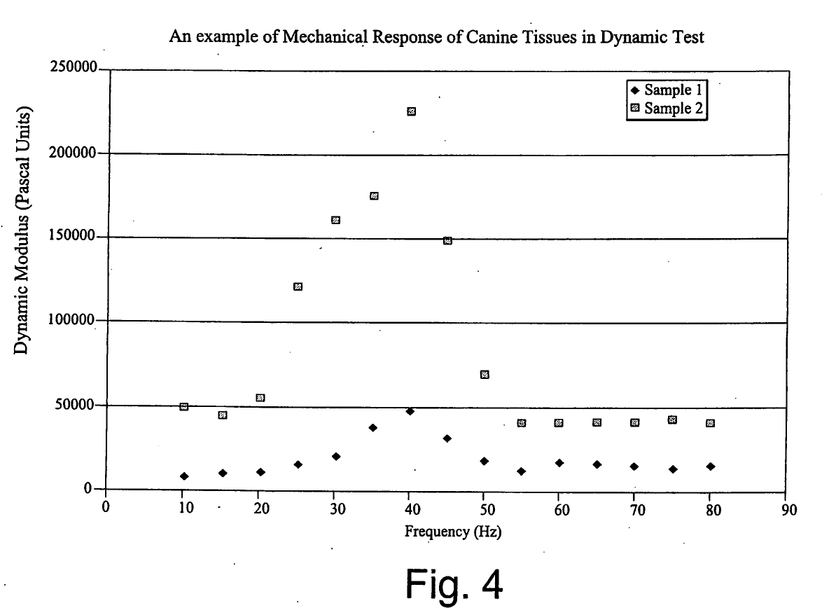

FIG. 4. shows two profiles of dynamic modulus as a function of frequency for distinct samples of canine prostate;

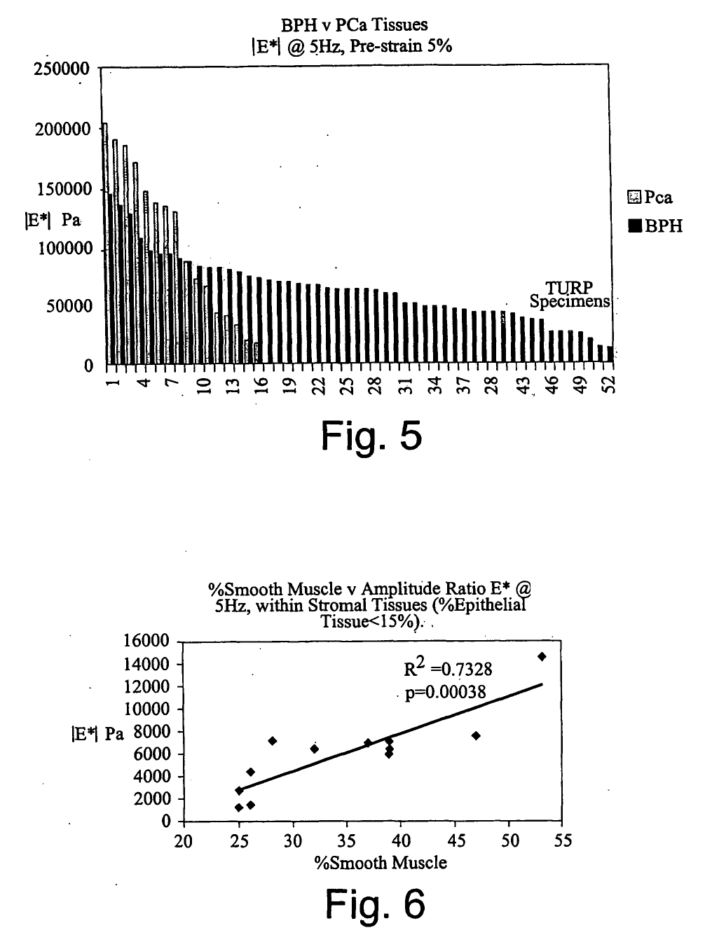

FIG. 5. shows values of Amplitude Ratio at a single frequency for a series of benign and cancerous human prostate tissue samples; and

FIG. 6 shows the relationship between Amplitude Ratio at a single frequency, and muscle content of a series of human prostate tissue samples.

FIG. 1 shows schematically a hydraulic piston based tissue quality measurement apparatus 1 with a probe portion 2 thereof inserted in the urethral passage 3 of a patient's penis 4 to examine the tissue quality of the prostate gland 5. In more detail the apparatus 1 generally comprises a relatively thick-walled partly flexible elongate hydraulic fluid tube 6, of a material such as polypropylene, which is substantially dimensionally stable so that a distal end portion 7 providing said probe portion 2 can be propelled along the urethral passage 3 by pushing on the proximal end portion 8, and so that hydraulic pressure can be efficiently transmitted along said tube 6 from the proximal 8 to the distal end portion 7 thereof.

In more detail, the probe portion 2 has a head portion 9 with two or more extending cylinder portions 10 in which are mounted respective pistons 11. A remotely controlled 12 distribution valve 13 is mounted in the head portion 9 for selectively connecting either one or more of the cylinder portions 10 to the hydraulic fluid tube 6. The head portion 9 is encased in a sheath 14 of a material such as silicone elastomer having resiliently deformable portions 15 closing the distal ends 16 of the cylinder portions 10 so that when the pistons 11 are forced outwardly by hydraulic fluid pressure, they are subjected to a return biasing force by said resiliently deformable sheath portions 15. The latter are also formed and arranged so as to be “mechanically transparent” i.e. they have small dynamic modulus compared with the tissue being measured.

The proximal end portion 8 of the hydraulic fluid tube 6 has a cylinder portion 17 mounting a drive piston 18 drivingly connected to a variable frequency periodic displacement drive 19 (such as an electromagnetically-operated linear displacement transducer) and provided with a displacement monitoring device 20 for recording the instantaneous displacement of the drive piston 18. The proximal end portion 8 also has a branch tube 21 provided with a pressure transducer device 22 connected thereto for monitoring hydraulic fluid pressure change as the drive piston 18 is displaced.

In use of the apparatus 1, when the drive piston 18 is displaced by the drive 19, hydraulic fluid is displaced along the tube 6 inducing a corresponding displacement of the head pistons 11 (due to the incompressibility of the hydraulic fluid). By previously calibrating the apparatus on the basis of a known displacement of the head pistons 11 for a given displacement of the drive piston 18, measuring displacement of the latter can be used to indicate the displacement of the former.

The displacement of the head pistons 15 will be resisted by the resiliently displaceable sheath portions 15 as well as the portion 23 of prostate tissue 5 displaced thereby. This will result in an increase in pressure in the hydraulic fluid measured by the pressure transducer 22, which depends on the elastic properties of the prostate tissue portion 23 displaced by the head piston 11, as well as those of the resiliently deformable sheath portions 15. Due to the damping characteristics of the displaced tissue portion 23, the increase in pressure (stress) will be out of phase with the displacement (strain) and the ratio of amplitudes and phase angle difference are used to determine the dynamic modulus.

FIG. 4 shows sample graphs obtained by in vitro probing of prostate tissue samples using a simplified form of the above apparatus, across a range of different frequencies from 10 to 80 Hz. As can be seen, the two tissues show distinct differences in the pattern of dynamic modulus vs frequency, with the greatest discrimination being at the frequency around 40 Hz. The pattern of dynamic modulus with frequency is correlated with tissue histology by a series of in vitro experiments.

### EXAMPLE 1

### Examination of Benign and Malignant Prostate Tissue Specimens Methods

52 fresh tissue specimens were collected from 10 patients undergoing Transurethral Resection of the Prostate (TURP) for benign prostatic obstruction (BPO). 16 fresh tissue specimens were collected from 3 patients undergoing TURP for obstruction due to carcinoma of the prostate (PCa). Individual tissue specimens underwent immediate mechanical testing, by applying a dynamic compressive strain to the samples using an electro-mechanical shaker, and the “dynamic modulus” or Amplitude Ratio (|E*|) was derived. |E*| values for benign and malignant tissue specimens were compared with single factor analysis of variance (ANOVA). Specimens were then fixed in formalin and embedded in paraffin wax. Epithelial tissues (ET) within sections from the processed tissues were then stained immunohistochemically with anti-PSA, and the size of individual glands within the stained ET measured with computerised image analysis. Individual gland size within benign and malignant tissue specimens were also compared with ANOVA.

Results

FIG. 5 shows simply values of amplitude ratio (the springiness component of dynamic modulus), sorted by magnitude, obtained by in vitro probing of human prostate tissue samples using a simplified form of the above apparatus, obtained at a single frequency of 5 Hz. As explained in more detail hereinbelow, this component of the dynamic modulus at this frequency is sufficient to differentiate the two types of tissue.

FIG. 6 shows sample values of amplitude ratio (the springiness part of dynamic modulus) obtained by in vitro probing of (non-cancerous) human prostate tissue samples using a simplified form of the above apparatus, again obtained at a single frequency of 5 Hz. In this case, all of the tissue samples probed were preselected to have a low content of glandular material (less than 15%). The smooth muscle content of the samples was determined by calculation of the stain area proportion in microscopic images stained selectively to show smooth muscle. As can be seen from FIG. 6, the histological make-up, even of the springy component of the tissue, has an effect on some aspect of the dynamic modulus, enhancing the quality of the diagnostic information available. In a more fully developed version of the device, a range of such information is likely to be used on the measured data in order to achieve a diagnosis based on a number of probe points around the gland.

More detailed analysis of the results shown in FIGS. 5 and 6, indicates that the |E*| (mean±SEM) of the 52 tissue specimens collected from patients undergoing TURP for BPO was 62±30 kPa. |E*| (mean±SEM) of the 16 tissue specimens collected from patients undergoing TURP for PCa was 105±66 kPa. Single factor ANOVA showed a statistically significant difference between the two groups (p=0.0004). The mean gland area within the ET of the 52 BPO specimens was 17000 μm², and within the 16 PCa specimens was 6500 μm². Again, ANOVA showed a statistically significant difference between the two groups (p=1.4*10⁻⁵).

FIG. 2 is a schematic view similar to that of FIG. 1 of a tissue quality measurement apparatus 24, with like parts being indicated by like reference numbers. The probe portion 2 of the apparatus 1 is mounted at the distal end portion 25 of a flexible substantially dimensionally stable conduit 26 connecting the probe portion 2 to a control unit 27.

The probe portion 2 houses, inside a sheath 14, a head portion 9 with a plurality of angularly distributed radially outwardly extending head elements 28 each of which has a piezoceramic element 29 sandwiched between an outwardly facing shoe element and a stress diaphragm 31. The piezoceramic elements 29 are electrically connected 32 to an electrical signal supply device 33 formed and arranged for applying an electrical signal at a range of different frequencies for activating the piezoceramic elements 29 so as to induce a predetermined displacement of the shoes 30. The force applied to the perturbed prostate tissue portion 23 is monitored by means of monitoring distortion of the stress diaphragm 31 using, for example, optical interferometry in known manner (see for example, Gander et al), the stress diaphragm 31 being coupled by optical fibers 34 to an optical interferometer device 35. The piezoceramic elements 29 could alternatively be provided with force transducers in the form of a micro-fabricated piezo-resistive strain gauge, mounted on the diaphragm to monitor its strain 29a.

In use of the apparatus, when an electrical signal is applied to the piezoceramic elements 29, a force is applied to the shoes 30 pressing them against the tissue portion 23. At the same time a corresponding reaction is experienced by the stress diaphragm 31. The displacement of the piezo-ceramic is determined by its characteristics and by the voltage applied an current drawn, whereas the strain in the reaction diaphragm measures the force applied. The force and displacement are used to determine dynamic modulus over a range of frequency of actuation is an analogous way to that described above.

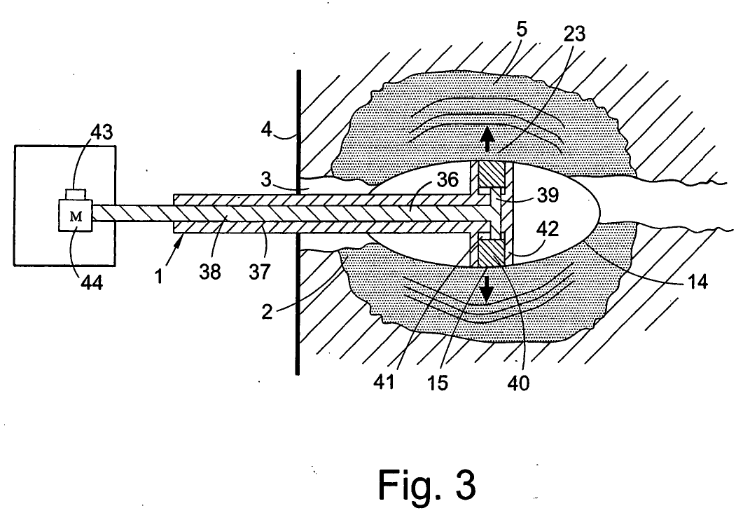

FIG. 3 is a schematic view similar to that of FIG. 1 of a tissue quality measurement apparatus 1, with like parts being indicated by like reference numbers. The probe portion 2 of the apparatus 1 is mounted at the distal end portion 36 of a sheathed 37 torsionally rigid flexible shaft drive transmission element 38. A cam element 39 is mounted at the distal end portion 36 of the drive shaft 38 for rotation by it, thereby periodically pushing outwardly pistons 40 mounted inside cylinders 41 provided in diametrically opposed radially outwardly extending head elements 42. As with the FIG. 1 embodiment the pistons 40 are captively retained inside the cylinders 41, by resiliently deformable sheath portions 15 of a head portion sheath 14, which also act as return springs for the pistons 40. The resistance to the outward displacement of the pistons 40, provided by the tissue portions 23, is monitored by means of a torque transducer 43 provided on the drive motor 44 of the drive shaft 38.

In use of this apparatus 1, the angular position of the rotary motor can be monitored by a shaft encoder or using the current-voltage characteristics of the motor. The angular position determines directly the displacement of the pistons. The force can be determined from the toque delivered by the motor, which can be measured either by its voltage-current characteristic or by the incorporation of a torque transducer in the drive shaft. Again, dynamic modulus can be determined from the time histories of the force and displacement.

### REFERENCES

Gander M J, MacPherson W N, Barton J S, Reuben R L, Jones J D C, Stevens R, Chana K S, Anderson S J, Jones T V. “Embedded micromachined fibre optic Fabry-Perot pressure sensors in aerodynamics applications”, IEEE Sensors 2002.

1. An apparatus suitable for use in investigating multi-phase biological tissue histology, which apparatus comprises a trans-ductally deployable probe mounting a periodically displaceable body of at least one tactile sensing device, said periodically displaceable body having an excitation frequency bandwidth in the range of from 1 Hz to 500 KHz, a maximum stroke length of less than 1 mm and a displacement force in the range from 0.01 N to 1 N, said displaceable body being provided with a displacement device having a displacement controller for controlling at least said excitation frequency, said displaceable body being coupled to a displacement monitoring device and a displacement force monitoring device, for monitoring the viscoelastic response of said biological tissue to periodic compression by said displacement force applied to said tissue by periodic displacement of said periodically displaceable body.

2. An apparatus according to claim 1 wherein said probe is formed and arranged so as to be trans-ductally deployable in at least one of the genito-urinary tract in males and females, the gastro-intestinal tract, the respiratory tract, and within the arterial and venous vasculature system.

3. An apparatus according to claim 1 for trans-urethral deployment, wherein and said probe has a diameter of not more than 5 mm.

4. An apparatus according to claim 3 wherein said probe has a length of from 1 to 3 cms.

5. An apparatus according to claim 1 wherein said displaceable body is actuated via at least one of: a pressurized fluid circuit, a mechanical drive system, and a piezoelectric actuator.

6. An apparatus according to claim 1 wherein said probe is mounted at the distal end of an elongate, trans-ductally deployable, deployment device.

7. An apparatus according to claim 6 wherein said displaceable body is actuated by a proximally mounted motor.

8. An apparatus according to claim 6 wherein said displaceable body is actuated by a distally mounted motor drivingly connected to the displaceable body via said elongate deployment device.

9. An apparatus according to claim 1 wherein said displaceable body comprises at least one micro-piston actuated via a pressurized fluid circuit.

10. An apparatus according to claim 1 wherein said displaceable body comprises at least one shoe mounted on a piezoelectric device sandwiched between said shoe and a stress detector element, formed and arranged for monitoring strain therein, thereby to determine the force applied by said displacement body to tissue contacted thereby in use of said apparatus.

11. An apparatus according to claim 1 wherein at least one of the area of the force-transmitting surface of the displaceable body, used to apply force to the tissue in use of the apparatus, and the magnitude of the force applied to the displaceable body, is formed and arranged so as to be user-adjustable.

12. An apparatus according to claim 1 wherein said displacement device incorporates an actuator whose position is controlled whereby in the control signal for said actuator may be used to monitor displacement of the displaceable body.

13. An apparatus according to claim 1 wherein a force detector is incorporated in at least one of the displaceable body displacement controller, the force source, and the displaceable body itself.

14. An apparatus according to claim 1 wherein is provided a displacement controller formed and arranged for application of selected ones of plurality of different excitation frequencies.

15. An apparatus according to claim 1 wherein is provided a displacement controller formed and arranged for controlling each of said excitation frequency and stroke length.

16. An apparatus according to claim 1, which includes a position control device for changing the position of the displaceable body within a body duct, in use of the apparatus, so as to successively bring it into contact with a plurality of different duct surface portions.

17. An apparatus according to claim 1 which includes a processing unit formed and arranged for processing displacement and displacement force data so as to generate at least one of dynamic modulus and Amplitude Ratio.

18. A method for producing a histological profile of a biological tissue adjacent a duct comprising the steps of:
a) providing an apparatus according to claim 1;
b) transductally inserting the probe of said apparatus to bring the periodically displaceable body of said probe into contact with the ductal surface of said biological tissue at a plurality of positions across said ductal surface;
c) subjecting said displaceable body to a periodic displacement at an excitation frequency bandwidth of from 1 Hz to 500 kHz, a maximum stroke of less than 1 mm and a displacement force in the range of from 0.01 N to 1 N so as to periodically force in the range of from 0.01 N to 1 N so as to periodically compress said biological tissue at said contact positions across said ductal surface;
d) monitoring the viscoelastic response of said tissue at each of said surface contact tissue positions to compression by said body; and
e) generating a profile of the viscoelastic response of the tissue across said ductal surface.

19. A method as claimed in claim 18 which includes the preliminary step of determining values of displacement frequency, displacement stroke length and displacement force suitable for histological profiling of the type of biological tissue to be profiled.

20. A method as claimed in claim 18, wherein said displacement body is contracted with a said plurality of tissue surface contact positions, which plurality is distributed axially and/or circumferentially of said duct.

21. A method of diagnosing a condition manifested by a histological abnormality in biological tissue adjacent a body duct comprising the steps of:
a) providing an apparatus according to claim 1;
b) trans-ductally inserting the probe of said apparatus to bring the periodically displaceable body of said probe into contact with the ductal surface of said biological tissue at successive ones of a plurality of positions across said ductal surface;
c) subjecting said displaceable body to a periodic displacement at an excitation frequency bandwidth of from 1 Hz to 500 kHz, a maximum stroke length of less than 1 mm and a displacement force in the range of from 0.01 N to 1 N so as to periodically compress said biological tissue at said contact positions across said ductal surface
d) monitoring the viscoelastic response of said tissue at each of said tissue surface contact positions to compression by said body;
e) generating a profile of the viscoelastic response of the tissue across said ductal surface; and
f) comparing said generated viscoelastic response profile with viscoelastic response profiles of such tissue having known histological characteristics.
